(19) United States
(12) Patent Application Publication
(10) Pub. No.: US 2010/0041942 A1
(43) Pub. Date: Feb. 18, 2010

(54) STABILIZER

(75) Inventor: Yuta OKADA, Hachioji-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/603,853

(22) Filed: Oct. 22, 2009

Related U.S. Application Data
(63) Continuation of application No. PCT/JP2008/058946, filed on May 15, 2008.

(30) Foreign Application Priority Data
May 16, 2007 (JP) 2007-130897

(51) Int. Cl. A61F 2/00

(52) U.S. Cl. 600/37

(57) ABSTRACT

A stabilizer makes contact with an organ thereby to restrict movement of the organ. The stabilizer includes a contact section that makes contact with an organ in a body cavity and an insertion section that includes the contact section at a top end of the insertion section, and further includes a lumen in which an endoscope for monitoring the organ or a surgical instrument with which a predetermined procedure is performed, or a guide wire remaining at a desired position near the organ is inserted, the insertion section being selectively switched to a pliable state and a rigid state.

### CROSS-REFERENCE TO RELATED APPLICATIONS

This is a Continuation Application of PCT Application No. PCT/JP2008/058946, filed May 15, 2008, which was published under PCT Article 21 (2) in Japanese.

This application is based upon and claims the benefit of priority from prior Japanese Patent Application No. 2007-130897, filed May 16, 2007, the entire contents of which are incorporated herein by reference.

### BACKGROUND OF THE INVENTION

1. Field of the Invention

The present invention relates to a stabilizer that makes contact with an organ such as the heart, to restrict movement of the organ.

2. Description of the Related Art

In cardiac surgery, sterna are cut (e.g., median sternotomy) to allow access to the chest cavity. In this case, a retractor is set in the opening made in the chest and separates widely the sterna and organs, to create a large opening. Surgical instruments are deployed through the opening, and cardiac surgery is then carried out.

One of most common forms of cardiac surgery is coronary artery bypass grafting (CABG). In CABG, occlusion in one or plural coronary arteries is bypassed by connecting a graft to a coronary artery on the downstream side of the occlusion. The technique for connecting a graft to a coronary artery is known as an anastomosis. As a graft, for example, a mammary artery which is cut from the chest wall is used. In this case, the upstream end of the mammary artery is left intact while the other downstream end of the mammary artery is connected to the coronary artery. Alternatively as a graft, an artery or vein cut from any part of the human body may be used. Also, alternatively, a piece of artificial blood vessel may be used as a graft. In this case, the upstream end of the piece grafted is connected to an artery such as the aorta while the other downstream end thereof is connected to the coronary artery. In this manner, occlusion in plural coronary arteries at various positions on the heart, such as at the front, side, or back of the heart, is bypassed by using plural grafts.

Since CABG is carried out while the patient's heart is stopped, the patient's blood is circulated by means of an artificial heart-lung machine.

However, CABG may alternatively be performed by stopping the patient's heart beating using a technique known as off-pump coronary artery bypass (OPCAB). Then, use of an artificial heart-lung machine can be avoided.

In OPCAB, the surface of the heart close to an anastomotic portion of a coronary artery is fixed by a special instrument called a stabilizer, with the heart beating. While a graft is being connected to a coronary artery by local fixation using the stabilizer, the anastomotic portion is kept as immovable as possible.

In the case of the stabilizer described above, for example as disclosed in Patent Document 1, a suction port is formed at a contact part to be brought into contact with an organ, and suctioning is performed with the contact part kept in contact with a desired portion of the heart, thereby to absorb the heart. Accordingly, movement of the heart is restricted.

Patent Document 1: Jpn. Pat. Appln. KOKAI Publication No. 11-197153

### BRIEF SUMMARY OF THE INVENTION

Accordingly, the present invention provides a stabilizer capable of bringing a contact section of a top end thereof into contact with a deep part (e.g., the back side) of an organ such as the heart through a small incision, thereby to restrict movement of the organ.

According to one aspect of the invention, there is provided a stabilizer that makes contact with an organ thereby to restrict movement of the organ, comprising: a contact section that makes contact with an organ in a body cavity; and an insertion section that includes the contact section at a top end of the insertion section, and further includes a lumen in which an endoscope for monitoring the organ or a surgical instrument with which a predetermined procedure is performed, or a guide wire remaining at a desired position near the organ is inserted, the insertion section being selectively switched to a pliable state and a rigid state.

### BRIEF DESCRIPTION OF THE SEVERAL VIEWS OF THE DRAWING

FIG. 1A is an exterior perspective view illustrating a structure of a stabilizer according to the first embodiment of the present invention;

FIG. 1B is a sectional view of a main part of the stabilizer;

FIG. 2 illustrates a method for inserting a stabilizer, according to the first embodiment;

FIG. 3 is an exterior perspective view illustrating a structure of a stabilizer according to the second embodiment of the invention;

FIG. 4 is an exterior perspective view illustrating a structure of a stabilizer according to the third embodiment of the invention;

FIG. 5 is an exterior perspective view illustrating a structure of a stabilizer according to the fourth embodiment of the invention; and

FIG. 6 is an exterior perspective view illustrating a structure of a stabilizer according to the fifth embodiment of the invention.

### DETAILED DESCRIPTION OF THE INVENTION

Hereinafter, best mode for carrying out the present invention will be described with reference to the drawings.

### First Embodiment

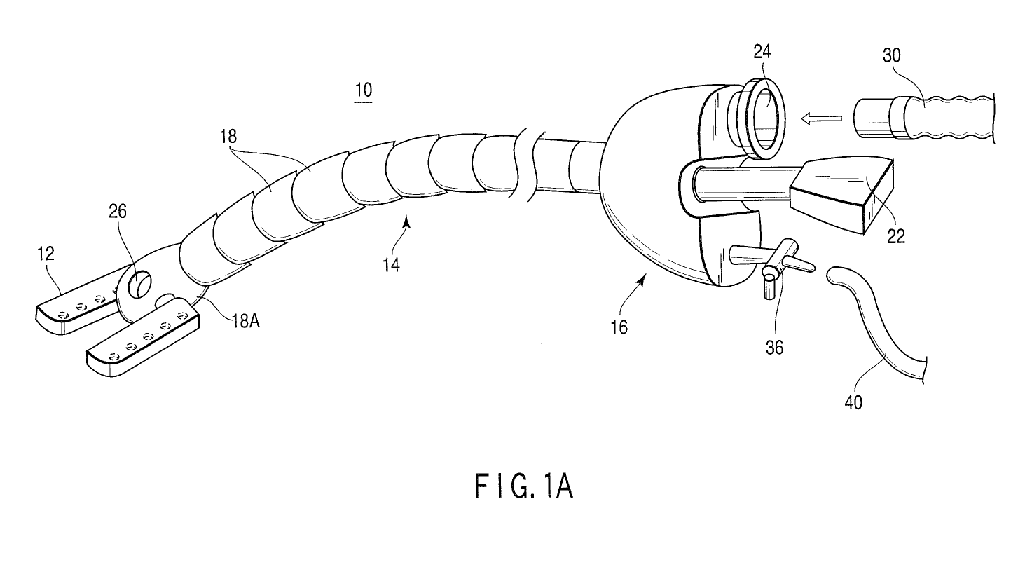

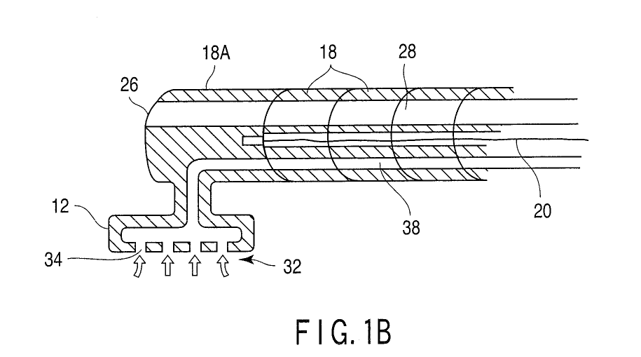

FIG. 1A is an external perspective view illustrating a structure of a stabilizer 10 according to the first embodiment of the invention. FIG. 1B is a sectional view of a main part of the stabilizer 10.

The stabilizer 10 includes: a contact section 12 which is brought into contact with a target organ; an insertion section 14 which has a top end where the contact section 12 is provided, and is capable of selectively switching a pliable (soft) state and a rigid (hard) state; and a manipulation section 16 which is provided at a rear end of the insertion section 14.

The insertion section 14 is formed of plural joint members 18. A joint lock wire 20 extends through inside of the joint members 18. An end of the joint lock wire 20 is fixed to a joint member 18A arranged at a distal end of the insertion section 14. The other end of the joint lock wire 20 is connected to a slider 22 provided on the manipulation section 16. As the slider 22 rotates, the insertion section 14 is switched between the pliable and rigid states. That is, tension of the joint lock wire 20 is strengthened by rotating the slider 22, and contact force between the joint members 18 is strengthened accordingly, thereby putting the insertion section 14 into the rigid state. That is, joints are locked thereby maintaining a bent state of the insertion section 14 which has been deformed in the pliable state. Further, the tension of the joint lock wire 20 is loosened by reversely rotating the slider 22, and the contact force between the joint members 18 is loosened accordingly, thereby putting the insertion section 14 into the pliable state. The joints are then allowed to be moved freely and bent into various bent states. Although the joint lock wire 20 is desirably extended through centers in the cross-sections of the joint members 18, the joint lock wire 20 may be slightly deviated from the centers.

Inside the joint members 18 of the insertion section 14, a lumen 28 is formed communicating from an inlet port 14 provided in the manipulation section 16 to an outlet port 26 provided at a top end of the joint member 18A provided at the distal end of the insertion section 14. The lumen 28 has a diameter which allows insertion of an endoscope 30 for monitoring an organ or a surgical instrument for performing a predetermined procedure on the organ.

Meanwhile, on a side of the contact section 12 where contact is made with an organ, an absorption section 32 for absorbing an organ is provided. The absorption section 32 includes plural absorption ports 34. Inside the joint members 18 of the insertion section 14, a suction path 38 is formed communicating from a valve 36 provided at the manipulation section 16 to each of the absorption ports 34 of the absorption section 32 provided at the contact section 12. A suction tube 40 connected to a suction pump not illustrated is connected to the valve 36.

Next, an insertion method for inserting a stabilizer 10 as described above into a desired portion is described.

Firstly, the endoscope 30 is inserted from the inlet port 24 provided at the manipulation section 16 as denoted by an arrow in FIG. 1A until the top end of the endoscope 30 protrudes from the outlet port 26. (The stabilizer 10 may desirably be pulled to a bottom side of the endoscope 30.)

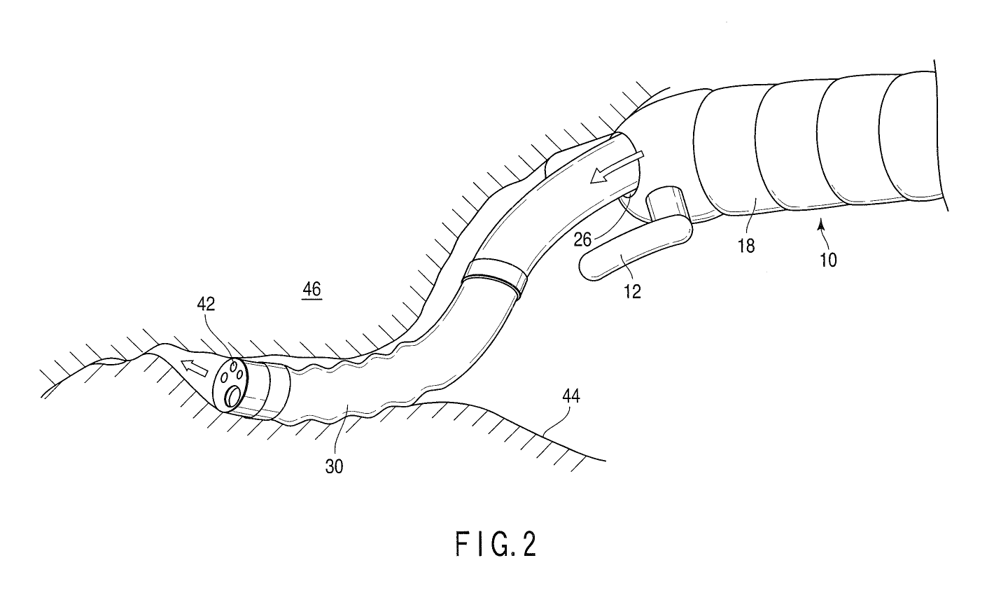

Then, as illustrated in FIG. 2, with the stabilizer 10 kept at this position, the endoscope 30 is further inserted through a hole made in a body cavity (not illustrated). While monitoring through a camera 42 provided at the top end of the endoscope 30, the endoscope 30 is inserted to a position of a target organ 44 to be brought into contact with the contact section 12 of the stabilizer 10, between the target organ 44, which is to be restricted from moving, and an adjacent organ (or a diaphragm) 46 adjacent to the target organ 44.

Thereafter, the insertion section 14 of the stabilizer 10 is put into the pliable state and then inserted along the endoscope 30. In this manner, the contact section 12 can be guided to a desired portion even if the stabilizer 10 is not provided with a camera. After the contact section 12 is thus guided to a desired position of the target organ 44, the slider 22 is rotated to switch the insertion section 14 into the rigid state. By opening the valve 36 to perform absorption, the contact section 12 is capable of absorbing the target organ 44 at a desired position thereof. Further, with the insertion section 14 maintained in the rigid state, the position and posture of the stabilizer 10 are adjusted so as to energize the contact section 12 toward the target organ 44. Movement of the target organ 44 is thereby restricted. At this time, force of holding the insertion section 14 in the rigid state can be adjusted by adjusting the slider 22, and pressure of the contact section 12 can be accordingly set to be adequate for the target organ 44 or the desired portion. When the contact section 12 of the stabilizer 10 is made to absorb the target organ 44, only the top end of the endoscope 30 may further be pushed to a deeper part, and bending of the endoscope 30 may then be inverted. In this manner, the contact section 12 can be found within a view field of the endoscope 30. Therefore, the contact section 12 can be made to absorb the target organ 44 at a desired position while monitoring the contact section 12 and the target organ 44 through the endoscope 30.

As has been described above, the stabilizer according to the first embodiment includes the lumen 28, which is open in the top end of the stabilizer and allows the endoscope 30 or a surgical instrument to be inserted through, and also includes an absorption section 32 at the top end of the stabilizer, which absorbs an organ. The insertion section 14 is configured to be switchable between the pliable and rigid states, and movement of the target organ 44 can be restricted by putting the insertion section 14 in the rigid state, with the target organ 44 absorbed by the insertion section 14. Accordingly, the endoscope 30 or surgical instrument can be inserted, in advance, to the back side of the target organ 44, and the stabilizer 10 can be inserted along the endoscope 30 or surgical instrument. The skin incision, therefore, need only be large enough to allow the stabilizer 10 to be inserted, and a surgical operation can be carried out with minimum invasiveness.

### Second Embodiment

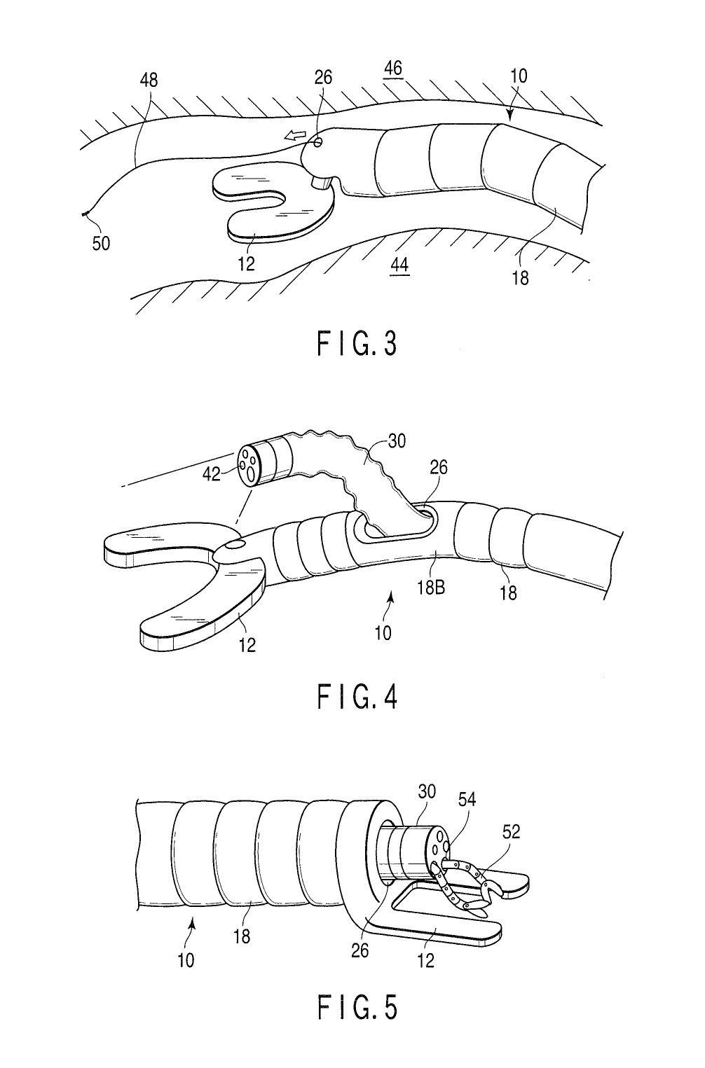

FIG. 3 is an external perspective view illustrating a structure of a stabilizer 10 according to the second embodiment of the invention.

In case of the stabilizer 10 according to the second embodiment, a lumen 28 is sized to have a diameter which allows a so thin member as a guide wire 48 to pass but does not allow an endoscope 30 or a surgical instrument to be inserted through, as in the first embodiment described above.

In this case, the guide wire 48 needs to hold in advance a front end 50 of the guide wire 48 near a desired position of a target organ 44, by using the endoscope 30 or surgical instrument. Further, a rear end (not illustrated) of the guide wire 48 extending outside a human body is inserted from an outlet port 26 of the stabilizer 10 and then extended out of an inlet port 24 through the lumen 28. Thereafter, the insertion section 14 of the stabilizer 10 is made pliable and inserted along the guide wire 48. In this manner, the contact section 12 can be guided to a desired position of the target organ 44 as in the first embodiment described above.

Otherwise, like the endoscope 30 described above in the first embodiment, firstly, the guide wire 48 may be inserted through the lumen 28 from the inlet port 24 and then extended from the outlet port 26 through the lumen 28. Then, the front end 50 of the guide wire 48 may be held near a desired position of the target organ 44 by further using the endoscope 30 or surgical instrument. The insertion section 14 may thereafter be made pliable and inserted along the guide wire 48.

Therefore, the stabilizer according to the second embodiment is capable of restricting movement of the target organ 44 by putting the insertion section 14 into the rigid state, with the target organ 44 absorbed by the insertion section 14 as in the first embodiment described above. Accordingly, the skin incision need only be of a size sufficient to allow the stabilizer 10 to be inserted, so that a surgical operation can be carried out with minimum invasiveness.

### Third Embodiment

FIG. 4 is an exterior perspective view illustrating a structure of the stabilizer 10 according to the third embodiment of the invention.

In case of the stabilizer 10 according to the third embodiment, an outlet port 26 from which a top end of an endoscope 30 or a surgical instrument is protruded is not provided at a top end of a joint member 18A positioned at a distal end but is provided on a side of at a joint member 18B in the middle.

Even with such a structure, the same operation and effect as the stabilizer 10 according to the first embodiment of the invention can be attained.

Further, by protruding the endoscope 30 from the side as in the present embodiment, the position of the contact section 12 of the stabilizer 10 can be observed so finely by the camera 42 is provided on the endoscope 30 that more accurate positioning and restriction of movement are possible.

### Fourth Embodiment

FIG. 5 is an exterior perspective view illustrating a structure of a stabilizer 10 according to the fourth embodiment of the invention.

In case of the stabilizer 10 according to the fourth embodiment, the position of an outlet port 26 and the shape of a contact section 12 are contrived, so that a procedure can be performed with the endoscope 30 protruded from the outlet port 26 through the lumen 28 while restricting movement of a target organ 44 by the contact section 12. Specifically, the endoscope 30 is provided with a forceps channel 54 for inserting a pair of biopsy forceps 52, communicating between a part which is positioned outside a human body when the endoscope 30 is inserted into the human body, and a top end of the endoscope 30. Hence, in the present embodiment, the endoscope 30 used for inserting the stabilizer 10 is directly used for a procedure.

Therefore, the stabilizer according to the fourth embodiment can exhibit the same operation and effect as the stabilizer 10 according to the first embodiment, and besides, there is no need to pull out the endoscope 30 used for inserting the stabilizer 10 from inside the human body after insertion of the stabilizer 10. Accordingly, labor on the part of the surgeon is saved, and the burden on the patient is reduced.

### Fifth Embodiment

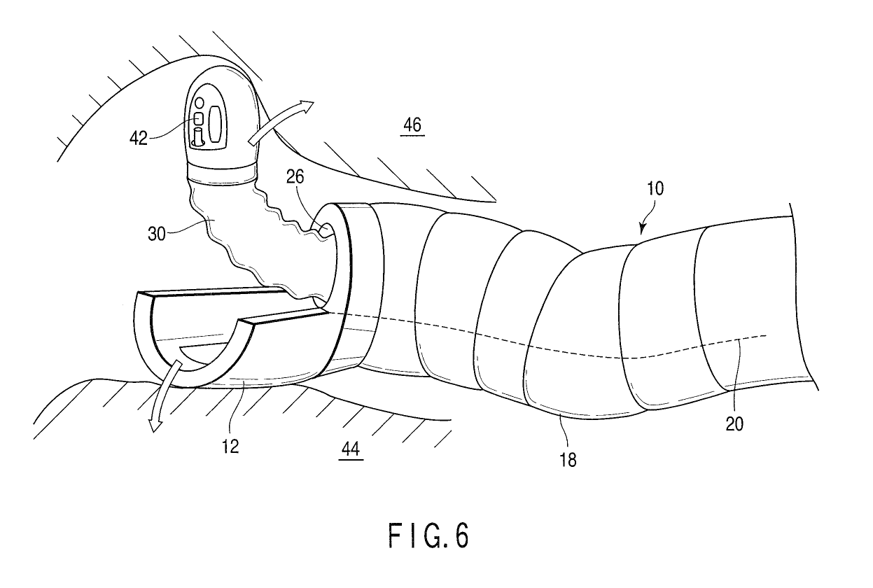

FIG. 6 is an exterior perspective view illustrating a structure of a stabilizer 10 according to the fifth embodiment of the invention.

The first to fourth embodiments each have a structure in which a contact section 12 is provided with an absorption section 32. The stabilizer 10 according to the fifth embodiment is configured to be capable of restricting movement by steadily bonding the contact section 12 to a target organ 44 without using such an absorption section 32.

Specifically, in the stabilizer 10 according to the fifth embodiment, the contact section 12 is provided extended toward the front to some extent from a top end of the insertion section 14. Further, a top end of an endoscope 30 protruded from an outlet port 26 provided at the top end of the insertion section 14 is pulled up toward an adjacent organ 46 facing a target organ 44 as denoted by an arrow in FIG. 6. Due to reaction thereof, the contact section 12 is pressed against the target organ 44, as denoted by another arrow in FIG. 6. In this state, a slider 22 may be rotated to put the insertion section 14 into a rigid state, and movement of the target organ 44 can thereby be restricted.

Further, if another camera 42 is provided on a side of the endoscope 30, an operative field can be monitored.

The present invention has been described above based on embodiments. However, the invention is not limited to the embodiments described above but may of course be variously modified or applied practically within the scope of the invention.

For example, the first, fourth, and fifth embodiments have been described referring to an example of inserting the endoscope 30 into the lumen 28. The endoscope 30, however, may alternatively be a surgical instrument such as a pair of biopsy forceps.

### Additional Notes

Inventions as configured below can be extracted from the specific embodiments described above.

(1) A stabilizer that makes contact with an organ thereby to restrict movement of the organ, comprising: a contact section that makes contact with an organ in a body cavity; and an insertion section that includes the contact section at a top end of the insertion section, and further includes a lumen in which an endoscope for monitoring the organ or a surgical instrument with which a predetermined procedure is performed, or a guide wire remaining at a desired position near the organ is inserted, the insertion section being selectively switched to a pliable state and a rigid state.

### Corresponding Embodiments

The first to fifth embodiments relate to the stabilizer described in (1). The stabilizer 10 in these embodiments corresponds the stabilizer described above; the target organ 44 corresponds to the organ described above; the contact section 12 corresponds to the contact section described above; the endoscope 30 corresponds to the endoscope described above; the guide wire 48 corresponds to the guide wire described above; the lumen 28 corresponds to the lumen described above; as well as the insertion section 14 corresponds to the insertion section described above.

### Operation and Effects

According to the stabilizer described in (1), the contact section at the top end is brought into contact with a deep part (such as a back side) of an organ such as the heart, through a small incision, and movement of the organ can thereby be restricted.

(2) The stabilizer described in (1), wherein the insertion section is put in the pliable state when inserted into the body cavity, and is put in the rigid state when restricting movement of the organ in the body cavity.

### Corresponding Embodiments

The first to fifth embodiments correspond relate to the stabilizer described in (2).

### Operation and Effects

The stabilizer described in (2) is switched to a pliable state when the stabilizer is inserted into a body cavity and to a rigid state when restricting movement. Accordingly, insertion into a body cavity is facilitated, and besides, movement of an organ can be steadily restricted.

(3) The stabilizer described in (1), wherein the contact section further has an absorption section that absorbs the organ.

### Corresponding Embodiments

The first to fourth embodiments correspond relate to the stabilizer described in (3). In these embodiments, the absorption section 32 corresponds to the absorption section described above.

### Operation and Effects

According to the stabilizer described in (3), an organ is absorbed by the absorption section. Therefore, the contact section can be constantly maintained in contact with the organ, and accordingly, stable restriction can be achieved.

What is claimed is:

1. A stabilizer that makes contact with an organ thereby to restrict movement of the organ, comprising:
a contact section that makes contact with an organ in a body cavity; and
an insertion section that includes the contact section at a top end of the insertion section, and further includes a lumen in which an endoscope for monitoring the organ or a surgical instrument with which a predetermined procedure is performed, or a guide wire remaining at a desired position near the organ is inserted, the insertion section being selectively switched to a pliable state and a rigid state.

2. The stabilizer according to claim 1, wherein the insertion section is put in the pliable state when inserted into the body cavity, and is put in the rigid state when restricting movement of the organ in the body cavity.

3. The stabilizer according to claim 1, wherein the contact section further has an absorption section that absorbs the organ.
